(19) United States
(12) Patent Application Publication
(10) Pub. No.: US 2012/0316249 A1
(43) Pub. Date: Dec. 13, 2012

(54) LYCOPENE FOR THE TREATMENT FOR METABOLIC DYSFUNCTION

(75) Inventors: Ivan Petyaev, Cambridge (GB); George Bash, Great Shelford (GB)

(73) Assignee: Cammedica limited, Cambridge (GB)

(21) Appl. No.: 13/450,017

(22) Filed: Apr. 18, 2012

Related U.S. Application Data
(63) Continuation of application No. 12/296,904, filed on Oct. 10, 2008, now abandoned.
(60) Provisional application No. 60/744,816, filed on Apr. 13, 2006.
(60) Provisional application No. 60/832,128, filed on Jul. 21, 2006.

(51) Int. Cl. A61K 31/01; A61P 3/04; A61P 3/10; A61P 5/50; A61P 9/12; A61P 1/16; A61P 3/06; A61P 3/00; A61P 5/26

(52) U.S. Cl. 514/762

(57) ABSTRACT

This invention relates to the treatment of metabolic dysfunction and disorders associated with metabolic dysfunction using lycopene compounds. Methods of treatment and uses of lycopene compounds in such methods are provided.

This invention relates to the methods and materials for the treatment metabolic dysfunction, including insulin resistance, glucose tolerance, hypertension, polycystic ovary syndrome, obesity, steatosis, chronic hepatitis and liver cirrhosis.

Metabolic abnormalities, such as insulin resistance, impaired glucose tolerance, hypertension, obesity and metabolic syndrome, have become increasingly common in the developed world and it is estimated that in America alone over 50 million people have such dysfunction. Metabolic dysfunctions are significant risk factors for the subsequent development of diabetes, cardiovascular disease, peripheral occlusive disease, cerebral and other forms of atherosclerosis.

Further development of effective treatments for these conditions would have a significant impact on the health of the world population.

The present inventors have found that lycopene has a dramatic effect on metabolic dysfunction in vitro, on animal models and in clinical trials in humans.

One aspect of the invention provides a method of treating a metabolic dysfunction comprising;
administering a lycopene compound in a therapeutically effective amount to an individual in need thereof.

Lycopene compounds may include lycopene and derivatives of lycopene which possess similar biological properties to lycopene. Lycopene is an open-chain unsaturated C₄₀ carotenoid of structure I (Chemical Abstracts Service Registry Number 502-65-8) which occurs naturally in plants such as tomatoes, guava, rosehip, watermelon and pink grapefruit.

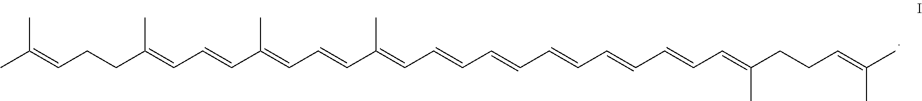

Lycopene for use as described herein may comprise one or more different isomers. For example, lycopene may comprise at least 10%, at least 20%, at least 30%, at least 40%, at least 50%, at least 60%, at least 70%, at least 80%, at least 90%, or at least 95% (Z)-isomers, (all-E)-isomers, or cis-isomers, such as 5-cis- or 9-cis- or 13-cis-isomers, which have improved bioavailability relative to trans isomers. Trans isomers may isomerise into cis forms in vivo, or during storage and processing.

Derivatives of lycopene which possess similar biological properties to lycopene may include, for example, carotenoids such as retinoic acid, synthetic acyclo-retinoic acid; or 4′-didehydrolycopene, 3,1′-(HO)2-gamma-carotene, 1,1′-(HO)2-3,4, 3′,4′-tetradehydrolycopene, and 1,1′-(HO)2-3,4-didehydrolycopene.

A lycopene compound for use as described herein may be natural i.e. obtained from a natural source, for example, extracted from a plant such as tomato or melon. A range of methods for extracting, concentrating and/or purifying lycopene compounds from plants are known in the art. For example, solvent extraction using ethanol, DMSO, ethyl acetate, hexane, acetone, soya or other vegetable oil, or non-vegetable oils may be employed.

Lycopene compounds for use as described herein may be synthetic i.e. produced by artificial means, for example, by chemical synthesis. A range of methods for chemical synthesis of lycopene and other carotenoids are known in the art. For example, a three-stage chemical synthesis based on the standard Wittig olefination reaction scheme for carotenoid synthesis may be employed, in which an organic solution of C₁₅ phosphonium methanesulfonate in dichloromethane (DCM) and an organic solution of C₁₀ dialdehyde in toluene are produced, and the two organic solutions are gradually combined with sodium methoxide solution and undergo a condensation reaction to form crude lycopene. The crude lycopene may then be purified using routine techniques, for example by adding glacial acetic acid and deionized water to the mixture, stirring vigorously, allowing the aqueous and organic phases to separate, and extracting the organic phase containing DCM and crude lycopene with water. Methanol is added to the organic phase and the DCM removed via distillation under reduced pressure. The crude methanolic lycopene solution is then be heated and cooled to crystalline slurry that is filtered and washed with methanol. The lycopene crystals may then be recrystallized and dried under heated nitrogen. Synthetic lycopene is also available from commercial suppliers (e.g. BASF Corp, NJ USA).

Synthetic lycopene may comprise an increased proportion of cis isomers relative to natural lycopene. For example, synthetic lycopene may be at up to 25% 5-cis, 1% 9-cis, 1% 13-cis, and 3% other cis isomers, whilst lycopene produced by tomatoes may be 3-5% 5-cis, 0-1% 9-cis, 1% 13-cis, and <1% other cis isomers. Since cis-lycopene has increased bioavailability relative to trans-lycopene, synthetic lycopene may therefore be preferred for some purposes.

Derivatives of lycopene as described above may be produced by chemical synthesis analogous to the synthesis described above or by chemical modification of natural lycopene extracted from plant material.

Lycopene compounds may be administered in any convenient form or formulation. Suitable formulations may facilitate or increase the absorbability of the lycopene compound and its bioavailability within the body. For example, a lycopene compound may be administered as a pharmaceutical composition comprising the lycopene compound, together with one or more pharmaceutically acceptable carriers, adjuvants, excipients, diluents, fillers, buffers, stabilisers, emulsifiers, preservatives, lubricants, or other materials well known to those skilled in the art and, optionally, other therapeutic or prophylactic agents. For example, in some embodiments, a lycopene compound may be administered as a pharmaceutical composition comprising the lycopene compound, together with isoflavones, for example soy isoflavones and/or vitamin C.

A suitable pharmaceutical composition may comprise a lycopene compound as the sole active component and may be formulated by admixing the lycopene compound together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990 and may include oils, for example vegetable oils, such as tomato oil, soya oil, or peanut oil, or non-vegetable oils, glycerol, gelatine, sucrose, glucose, ascorbyl palmitate, corn starch and silicon dioxide. Suitable emulsifiers include polysorbate 80.

Suitable stabilisers may include sucrose ester, lecithin, antioxidants such as dl-a-tocopherol and ascorbyl palmitate, flavanoids, cellulose, waxes, mannitol, shellac, talc, calcium phosphate, magnesium stearate, and arabic or acacia gum.

For example, lycopene may be formulated into beads, tablets or other solid bodies, containing about 10% lycopene, 1.5% and 5.0%, respectively, of the antioxidants dl-a-tocopherol and ascorbyl palmitate, in addition to carrier substances such as vegetable oil, fish gelatine, sucrose and corn starch.

Suitable formulations of lycopene are commercially available and include LycoVit™ 10 Percent, LycoVit™ 10 Cold Water Dispersion (CWD), and Lycovit™ Dispersion 20 Percent (all BASF Corp, NJ, USA), Lyc-O-Mato™ (LM), LycoBeads™, and Tomato-O-Red™ (Dalidar Pharma, LycoRed Ltd UK).

In some embodiments, a lycopene compound may be formulated with a solubilising agent. Solubilising agents include hydrophilic compounds that are soluble in aqueous solution and may also be insoluble in organic solvents. Suitable hydrophilic solubilising agents include soluble proteins in particular lactoproteins, such as casein, beta-lactoglobulin, alpha-lactalbumin, and serum albumin. Conveniently, whey protein may be used as solubilising agent. Whey protein is a collection of globular proteins isolated from whey, which is a by-product of cheese manufacture from cow's milk. Whey protein is a mixture of beta-lactoglobulin (˜65%), alpha-lactalbumin (˜25%), and serum albumin (˜8%), which are soluble in their native forms, independent of pH. Lycopene formulations with whey protein (termed ‘lactolycopene’) are known in the art (see for example, Richelle et al J. Nutr. 132:404-408, 2002 and EP-B-1289383 (PCT/EP01/06145)) and are available commercially (INNEOV, L'Oréal (UK) Ltd, London). A process for the preparation of a lycopene formulation with whey protein may comprise admixing the whey protein with lycopene under conditions sufficient to form a mixture. For example, whey protein may be dissolved in water, and lycopene may be dissolved in a solvent, such as acetone, ethanol or isopropanol, the two solutions combined and the solvent evaporated to form a lacto-lycopene composition.

Alternatively, the composition may be formed by mixing the lycopene with a solvent to form a first mixture, mixing the first mixture with the whey protein in the form of a powder or aqueous solution to form a second mixture and evaporating the solvent from the second mixture to produce a composition as a dispersion. Suitable solvents include acetone, ethanol and isopropanol. Conveniently, a solvent:water ratio of the order of 60:40 by volume may be employed when the aqueous phase is mixed with the solvent. A composition may comprise up to 50% lycopene compound and up to 90% whey protein.

The dispersion may optionally be heated treatment to produce a gel or dried by spraying or by lyophilisation to produce a powder.

The composition may then be formulated with carriers, excipients, stabilisers and emulsifiers as described above. For example, a suitable lactolycopene formulation may comprise 2% (w/w) lycopene, 20% (w/w) whey protein, 50.5% (w/w) microcrystalline cellulose, 5% (w/w) silicon dioxide, 3% (w/w) polysorbate 80 and 1.5% (w/w) soy lecithin.

A lactolycopene formulation for use as described herein may be obtained or obtainable as described above.

Metabolic dysfunctions which might be treated with lycopene as described herein may include obesity, insulin resistance, reduced glucose tolerance, polycystic ovary syndrome (PCOS), hypertension, liver disorders, such as steatosis, chronic hepatitis, liver fibrosis and cirrhosis, and metabolic syndrome.

In some embodiments, an individual treated in accordance with the present methods is not diabetic (i.e. is not suffering from type 1 or type 2 diabetes) and/or is not suffering from an atherosclerotic condition, such as CHD, stroke or peripheral vascular disease.

Obesity is a condition characterised by excess body fat. For example, an obese individual may have a body mass index (BMI: Mass/Height²) of greater than 30. Obesity is a risk factor for a range of medical conditions, including diabetes and cardiovascular disorders such as atherosclerosis, ischaemic (coronary) heart disease, myocardial ischaemia (angina) and stroke. Obesity may include abdominal obesity, which is commonly defined as waist circumference more than 94 cm for European men and more than 80 cm for European women, more than 90 cm for South Asian men and more than 80 cm for South Asian women, and more than 85 cm for Japanese men and more than 90 cm for Japanese women. The methods described herein may be useful in weight control and the treatment of obesity.

Insulin resistance is the inability of cells, tissues or the whole body to show a physiological response to a given amount of insulin. As a result, higher insulin amounts are required to achieve the physiological effect of insulin compared to controls. Insulin resistance is present in 25% of the non-diabetic population, with an estimated conversion rate from an insulin resistant state to type 2 diabetes of 2-12% per year.

Reduced glucose tolerance occurs when a glucose concentration of greater than 7.8 mmol persists in the plasma after 120 min of the oral glucose tolerance test. Reduced glucose tolerance is associated with insulin resistance and hyperglycemia.

Polycystic ovary syndrome (PCOS) is an endocrine disorder that affects 5-10% of women, in which cysts in the ovary interfere with normal ovarian cycle of hormone production. PCOS is often associated with insulin resistance, compensatory hyperinsulinaemia, metabolic syndrome, obesity, and hyperandrogenaemia.

Hypertension is characterised by a consistently elevated blood pressure which exceeds 140 over 90 mmHg (>140 systolic pressure; >90 diastolic pressure).

Liver disorders include steatosis, chronic hepatitis, liver fibrosis and cirrhosis. Liver steatosis is characterised by an abnormal lipid accumulation in the liver which may be caused by reduced oxidation of fatty acids and/or decreased synthesis and release of lipoproteins. Steatosis may subsequently transform into liver fibrosis and cirrhosis. Liver fibrosis is characterised by the growth of fibrous tissue in the liver. Fibrosis can lead to cirrhosis, in which the liver has reduced function and becomes permanently scarred, fibrous, and fatty.

Chronic hepatitis is an inflammation of the liver which may be caused by bacterial or viral infection, parasitic infestation, alcohol, drugs, toxins, or transfusion of incompatible blood

Metabolic syndrome is a condition which is characterised by obesity and one or more of; insulin resistance, high blood pressure, high blood triglyceride levels and/or low HDL cholesterol levels in the blood. For example, the IDF (International Diabetes Federation) defines metabolic syndrome as: abdominal obesity plus any two of the following: raised triglyceride levels (above 1.7 mmol/L); reduced HDL cholesterol (below or at 0.9 mmol/L in men and below or at 1.1 mmol/L in women); raised blood pressure (above 130/85) and raised fasting plasma glucose (above 5.6 mmol/L).

Metabolic dysfunctions, such as metabolic syndrome, may also be associated with a range of conditions including hypercholesterolaemia, low HDL cholesterol (for example, <0.9 mmoll⁻¹, men; <1.0 mmoll⁻¹, women), hypertriglyceridaemia (for example, plasma TAG's ≧1.7 mmoll⁻¹) and microalbuminuria (for example, urinary albumin excretion rate ≧20 μg min⁻¹; albumin: creatinine ratio ≧30 mg min⁻¹).

The methods described herein may be useful in the treatment of a condition which is associated with metabolic dysfunction. For example, a method of treating a condition which is associated with metabolic dysfunction may comprise;
administering a lycopene compound to an individual in need thereof.

A condition associated with metabolic dysfunction may include hypercholesterolaemia, low HDL cholesterol, hypertriglyceridaemia, and microalbuminuria.

Methods of the invention may also have prophylactic applications. For example, a method of preventing or delaying the onset of metabolic dysfunction may comprise administering a lycopene compound to an individual in need thereof.

An individual suitable for undergoing a method of preventing or delaying the onset of a metabolic dysfunction as described herein may be at risk of or susceptible to metabolic dysfunction, for example the individual may have one or more risk factors associated with the onset of metabolic dysfunction.

A method described herein may comprise administering the lycopene compound in combination with a statin (i.e. a 3-Hydroxy-3-methylglutaryl Coenzyme A (HMG Co A) reductase inhibitor).

Suitable statins include pravastatin (PRAVACHOL™), lovastatin (MEVACOR™), simvastatin (ZOCOR™), cerivastatin (LIPOBAY™), fluvastatin (LESCOL™), atorvastatin (LIPITOR™), mevastatin and rosuvastatin (Crestor™). Other suitable statins are known in the art and may be readily identified by using HMG-CoA reductase assays which are well known in the art. Examples of such assays are disclosed in U.S. Pat. No. 4,231,938.

Another aspect of the invention provides the use of a lycopene compound in the manufacture of a medicament for use in the treatment of a metabolic dysfunction or a condition associated with metabolic dysfunction as described above, or preventing or delaying the onset of a metabolic dysfunction or a condition associated with metabolic dysfunction.

Another aspect of the invention provides a lycopene compound for use in the treatment of a metabolic dysfunction or a condition associated with metabolic dysfunction and a lycopene compound for use in preventing or delaying the onset of a metabolic dysfunction or a condition associated with metabolic dysfunction.

A composition or medicament comprising a lycopene compound may further comprise a statin (i.e. a 3-Hydroxy-3-methylglutaryl Coenzyme A (HMG Co A) reductase inhibitor.

Examples of suitable statins are described above. Other statins useful in the methods of the present invention will be apparent to the skilled person.

Another aspect of the invention is a kit comprising a lycopene compound and a statin for use in combination to treat metabolic dysfunction.

A kit may further include one or more other articles and/or reagents for performance of the method or the invention, such as means for administering the lycopene compound alone or in composition with other compounds with a syringe (such components generally being sterile).

The kit may further include instructions for carrying out all or part of the method of the invention, e.g. the dosage regime for the lycopene compound and/or the statin.

Yet another aspect of the invention is use of a combination of a lycopene compound and a statin in the manufacture of a medicament for the treatment of metabolic dysfunction or a condition associated with metabolic dysfunction as described above, or preventing or delaying the onset of a metabolic dysfunction or a condition associated with metabolic dysfunction.

Formulations of lycopene compounds for use in the present methods may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, dispersions, emulsions, elixirs, syrups, tablets, lozenges, granules, powders, capsules, cachets, beads, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as, an oil-in-water liquid emulsion or a water-in-oil liquid emulsion; as a bolus; as an electuary; or as a paste. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Administration is preferably in a “therapeutically effective amount”, this being sufficient to show benefit to the individual. It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of lycopene is in the range of about 100 μg to about 250 mg per kilogram body weight of the subject per day. Where the active compound is a salt, an ester, pro-drug, or the like, the amount administered is calculated on the basis of the parent compound and so the actual weight to be used is increased proportionately.

A composition comprising a lycopene compound such as lycopene may be administered alone or in combination with other treatments, either simultaneously or sequentially dependent upon the condition to be treated.

A lycopene compound may be comprised in a food product. For example, it may be comprised in bread, cereal, biscuits, butter, spreads (e.g. margarine), cheese, yogurts or beverages. Other suitable food products will be apparent to a person skilled in the art.

The lycopene compound may be mixed with the ingredients of the food product prior to cooking (e.g. baking) and/or added to the food product after cooking. The data herein show that lycopene compounds can be incorporated into food products without loss of food quality and remain active after being cooked into food products.

Preferably a heterologous lycopene compound is comprised in a food product. For example, a synthetic lycopene compound, as described above, or a natural lycopene compound that is not naturally present in the food product. For example, a lycopene compound from a fruit or vegetable may be incorporated into bread or cereal.

Thus, a still further aspect of the invention is a food product comprising a lycopene compound for treating metabolic dysfunction, wherein the lycopene compound is not naturally present in the food product.

The food product may be supplemented with or fortified by the lycopene compound. In some preferred embodiments, the food product is supplemented with a lycopene compound which is formulated with whey protein as described above (‘lactolycopene’).

A method of making a food product for treating or delaying or preventing the onset of metabolic dysfunction is also provided, said method comprising:
(i) providing a food product ingredient,
(ii) mixing a lycopene compound with said ingredient
(iii) formulating said ingredient and said lycopene compound into a food product.

Various further aspects and embodiments of the present invention will be apparent to those skilled in the art in view of the present disclosure. All documents mentioned in this specification are incorporated herein by reference in their entirety.

Certain aspects and embodiments of the invention will now be illustrated by way of example and with reference to the figures described below.

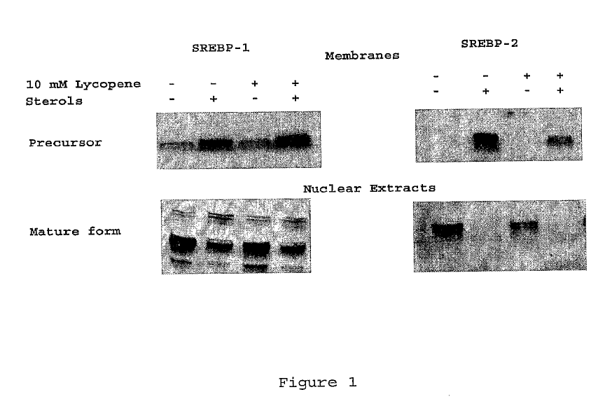

FIG. 1 shows the effect of lycopene on the amount of SREBP-1 and -2 in membrane and nuclear fractions of HepG2 cells.

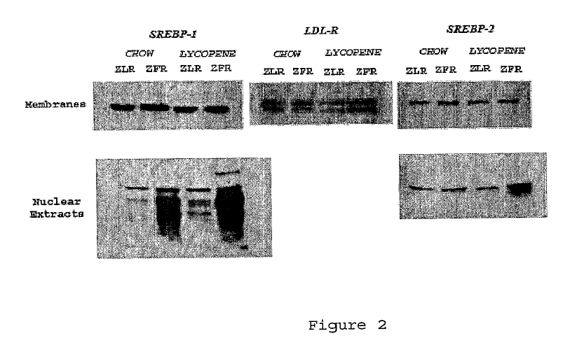

FIG. 2 shows immunoblot analysis of SREBP-1, -2 and LDL-R in liver membrane and nuclear extracts of Zucker Fatty (ZFR) and Lean (LZR) rats after lycopene treatment.

Table 1 shows mRNA changes in HepG2 cells incubated with 10 μM lycopene in the presence and absence of sterols.

Table 2 shows mRNA changes in Rat primary hepatocytes incubated with 10 μM lycopene in absence and presence of 100 nM insulin.

Table 3 shows the physiological parameters of Zucker Fatty (ZFR) and Zucker Lean (ZLR) Rats treated with 0.2% lycopene diet (M±m from 4 measurements in 4 rats).

Table 4 shows mRNA changes in the livers of Zucker Fatty (ZFR) and Zucker Lean (ZLR) rats treated with 0.2% lycopene Diet.

Table 5 shows the effect of Lactolycopene, LL, on the level of serum lipids in human volunteers.

Table 6 shows the effect of LL on the mean level of serum lipids in human volunteers following two months of treatment with LL.

Table 7 shows the effect of Lyc-O-Mato™, LM, on the level of serum lipids in human volunteers.

Table 8 shows the effect of LM on the mean level of serum lipids in human volunteers following two months of treatment with LM.

Table 9 shows the effect of LL on glucose concentration in the serum of diabetic patients.

Table 10 shows the effect of LM on glucose concentration in the serum of diabetic patients.

Table 11 shows the effect of Lactolycopene, LL, on the level of serum insulin in human volunteers.

Table 12 shows the effect of LL and LM on the body mass of human volunteers.

Table 13 shows the effect of Lipitor-Liprimar (Atorvastatin) on the level of serum lipids in human volunteers.

Table 14 shows in vitro anti-oxidant activity of different baked food matrixes (bread loaves and scones) that comprise lactolycopene (LL).

Table 15 shows the effect of lactolycopene on parameters of lipid metabolism in human blood serum.

Table 16 shows the effect of lactolycopene on glucose level in human plasma.

### Experiments

### Materials

Standard molecular biology techniques were used. Total RNA from cultured cells and rat liver was extracted using RNA-STAT-60 isolation reagent (Tel-Test, Inc, USA). SYBR Green qPCR reagent system (Invitrogen, USA) was used for quantitative RNA analysis. Plasma insulin levels were measured using ELISA Kit (Linco Research, USA), plasma glucose—colorimetrically.

Unless otherwise stated, lycopene was the whey protein associated lactolycopene formulation (LL) which was used in a commercial form (INNEOV, L'Oreal (UK) Ltd, London), which additionally contains as additives soy isoflavones and vitamin C, or in an isolated form (INDENA) produced by admixing 20 g tomato extract (containing 10% lactolycopene) with 20 g whey protein, 50.5 g microcrystalline cellulose, 5 g silicon dioxide, 3 g polysorbate 80 and 1.5 g soy lecithin to produce 100 g of lactolycopene formulation.

Lactolycopene formulations were prepared as described in EP1289383.

Other general reagents were obtained from Sigma (USA).

### Cell Culture

Monolayers of human hepatoma cell line—HepG2 were set up at density 1 million cells/100 mm dish and cultured at 6% CO2 at 37C. in DMEM supplemented with 10% FCS with penicillin (100 units/ml) and streptomycin (100 μg/ml). 48 hours later, cells were washed twice with PBS and switched to DMEM, containing 5% lipoprotein-deficient serum, 50 μM mevalonate and 50 μM compactin (lactone form) in the absence or presence of sterols (1 μg/ml 25-hydroxycholesterol and 10 μg/ml cholesterol). Half of the dishes also received INNEOV lactolycopene dissolved in tetrahydrofuran up to final concentration of 10 μM. Another half received aliquot of solvent alone. The cells were harvested for immunoblot analysis and RNA extraction 18 hours later.

Primary hepatocytes were isolated from rat liver of non-fasted Spraque-Dawley rats by collagenase digestion method. Under halothane anesthesia each liver was perfused in situ through portal vein subsequently with Liver perfusion medium and Liver Digest Medium (Gibco/BRL, USA). Livers were excised, hepatocytes dissociated and plated onto 100 mm collagen I-coated dishes (Becton Dickinson Labware, USA) at density 6 million cells per dish in DMEM containing 5% Fetal Calf Serum with penicillin (100 units/ml) and streptomycin (100 μg/ml). After 3 hours attachment the cells were switched to serum-free DMEM, supplemented with 100 nM dexamethasone, 1 nM insulin, 100 nM triiodthyronine, 100 units/ml penicillin, 100 μg/ml streptomycin. Viable cells were counted trypan blue exclusion test. Hepatocytes with at least 85% of viable cells were used for studies. After overnight incubation, addition of lactolycopene (INNEOV) solution in tetrahydrofuran up to final concentration 10 μM were made in presence or absence 100 nM insulin. Control dishes received tetrahydrofuran alone. RNA isolation has been done 18 hours after supplementation of hepatocytes with lycopene and/or insulin.

### Animals and Treatments

Zucker Fatty rats (fa/fa) and their lean littermates were used in the experiments at the age 8-9 weeks. Rats were housed in colony cages, maintained on a 12 h light/12 h dark cycle and fed with regular chow diet. After 10 days of adaptation to the colony environment, rats were switched to regular chow diet supplemented with 0.2% of Lactolycopene (INNEOV). Animals were fed ad libitum with 24 hour access to the food. Daily monitoring did not show substantial difference in the food intake between lycopene treated/untreated groups of rats. After 8 days of the dietary treatment, rats were killed under halothane anaesthesia. Blood samples were collected and body/liver weights recorded.

### RNA Analysis

Total RNA from cultured cells and rat liver was extracted and 2 μg of RNA from each sample was used to synthesize cDNA and RT-PCR reactions were performed in triplicates. Primers for each individual genes were designed using public database Genebank. Real CT values for each RNA sample incubated with specific primers were referred to CT values of housekeeping genes—GAPDH or 36B4 (internal controls), whose absolute CT values are shown for each experiment. Normalized CT value for each gene, observed in HepG2 cells in absence of sterols and were valued as 1.00. Changes of mRNA levels in other groups were referred to 1.00 and represent fold changes over the control value (1.00).

### Immunoblotting

For immunoblot analysis nuclear extracts and membrane fractions were prepared from cultured cells and rat livers. Cells were disrupted in hypotonic buffer (10 mM Hepes-KOH at pH 7.4) passing the cells through 22.5 gauge needle. Nuclear extracts and membrane fractions from rat liver were isolated as described elsewhere.

### Clinical Trials

The objective of the trial was to study whether lactolycopene itself, regardless of its particular formulation, can affect metabolic parameters in humans. For this trial, 14 clinically healthy volunteers with were recruited. 12 of them had one or another form of dislipidaemia, 3 of them had in addition elevated fasting glucose, 1 had only elevated fasting glucose and 1 neither of these metabolic disorders. To 7 volunteers LL was administered in dose of 300 mg, or 6 mg of HPLC lycopene, to other 7 the dose was 800 mg of LL, or 14 mg of HPLC lycopene. The product was administered for two months, once a day orally with food. The blood of the volunteers before the trial and in the end was tested and compared.

### Results

### EXAMPLE 1

Human hepatoma cell line HepG2 has been used to reveal effect of lactolycopene on transcriptional regulation of lipid metabolism in the cultured cells. As can be seen from Table 1, in all groups of the experiment mRNA level for basic housekeeping gene remained almost unchanged and varied from 17.54 to 17.73. That validates absence of drug toxicity, RNA degradation and healthy status of the cell monolayers.

As can be seen from the table 1, addition of 10 μM Lactolycopene did not affect significantly SREBP-1 mRNA level in presence or absence sterols. Reduction of SREBP-2 mRNA in response to sterols was the same in both groups regardless of lactolycopene presence.

Sterols are known to block transcription of SREBP-2 in the cultured cells, causing subsequent decline in cholesterol biosynthesis and mRNAs for cholesterologenic enzymes. Table 1 shows, that addition of the sterols to HepG2 cells efficiently reduced mRNAs for SREBP-2 and major enzymes of cholesterol biosynthesis—FPPS, HMG-CoA Red and HMG-CoA Syn, However, there is an obvious decline in SREBP-2 mRNA level even in absence of the sterols (42% reduction) in HepG2 cells incubated with 10 μM lactolycopene alone. Reduced SREBP-2 level in presence of Lactolycopene did not affect most of the cholesterologenic and lipogenic mRNAs. However, most of the lipogenic genes (FAS, ACC, HMG-CoA Syn and HMG-CoA Red) showed slightly higher sterol sensitivity in presence of lactolycopene. Lactolycopene alone decreases mRNA for SCAP, without potentiation of sterol effect on SCAP mRNA.

The only SREBP-1 downstream gene, affected by lactolycopene was INSIG-1 whose mRNA dropped almost by ⅔ of basal level. The immunoblot data (FIG. 1) confirm and explain mRNA results. In absence of the sterols, there is no detectable precursor form of SREBP-1 and -2 in membrane fractions of HepG2 cells. However, when cleavage of SREBP-1 stops in presence of sterols, amount of SREBP-1 protein in presence of lactolycopene is obviously higher, which reflects ˜1.4 SREBP-1 mRNA upregulation. Immunoblot results also confirm the decline in SREBP-2 mRNA level seen in HepG2 cells incubated with lactolycopene—amount of the SREBP-2 precursor and mature forms obviously lower in HepG-2 cells incubated with lactolycopene.

In a separate set of the studies, conducted using the same protocol, we have confirmed and reproduced inhibitory effect of lactolycopene on SCAP and INSIG-1 mRNAs.

Lactolycopene reduces mRNAs for major cholesterologenic transcription factor SREBP-2 and insulin-inducible gene-1 (INSIG-1), conferring slightly higher sterol-sensitivity for lipogenic genes in human hepatoma cell line HepG2. Taken together, our results provide indication that lactolycopene affects some transcriptional mechanisms of lipogenesis and possibly liver-specific insulin response in human hepatocytes.

### EXAMPLE 2

Due to noticeable effect of lycopene on some insulin-regulated genes in HepG2 cells (SREBP-1 and INSIG-1), the rat primary hepatocyte system and the insulin treatment protocol were chosen to further investigate the physiological properties of lactolycopene. As can be seen from Table 2, treatment of primary rat hepatocytes with 10 μM lactolycopene did not much affect absolute CT values for a major housekeeping gene—36B4. This confirms absence of toxic effects, acceptable viability of primary hepatocytes and good RNA quality extracted from the cells.

It is evident from the results, that insulin selectively up-regulates mRNA for a major liver-specific lipogenic transcriptional factor—SREBP-1c, whose corrected CT value went up over the basal level up to 56.5 folds. lactolycopene itself enhanced basal SREBP-1c level by 4 fold. Meanwhile, SREBP-1c induction with insulin was diminished by addition of lactolycopene as much as twice (only 27.5 folds).

Similarly to the HepG-2 study, the presence of lactolycopene was found to reduce mRNA for a major cholesterologenic transcriptional factor—SREBP-2, especially in the presence of insulin. The blunted response of SREBP-1c to insulin, as well as the reduced level of SREBP-2 to insulin in presence lactolycopene, appears to be functionally significant and has metabolic consequences in the primary hepatocytes. In particular, the presence of lactolycopene completely abolished insulin response of major lipogenic mRNAs—FAS, SCD-1, ACL and INSIG-1, known to be target genes for SREBPs.

These mRNA levels were corrected to the near control values upon lactolycopene treatment.

Another important feature of lactolycopene action is regulating activity towards major gluconeogenic enzymes—PEPCK and G-6-Pase, whose mRNA levels dropped correspondingly to 66.0% and 70% with lactolycopene alone. Insulin brought down PEPCK mRNA almost by 90% and showed the same potency in the presence of lactolycopene. In contrast, G-6-Pace response to insulin was not that dramatic. Remarkably, incubation with lactolycopene conferred higher insulin sensitivity to G-6-Pase. That mRNA was just slightly reduced with insulin alone (to 90% of the control level), but decreased more significantly in presence of both—lactolycopene and insulin (to 62% of the control level).

Lactolycopene enhanced insulin sensitivity of some other insulin target genes, which are not related to gluconeogenesis. There is more profound negative effect of insulin on INSIG-2, IRS-2 and IGFBP-1 mRNAs when primary hepatocytes were exposed to insulin in presence of lactolycopene.

Lactolycopene therefore reduces transcriptional lipogenic response and SREBP-2 mRNA in presence of insulin, provides higher insulin-sensitivity for gluconeogenic enzymes in rat primary hepatocytes.

### EXAMPLE 3

To further investigate the mechanism of lactolycopene effect on hepatic metabolism, we have conducted a study using Zucker Fatty Rats (ZFR), known to display most important features of the metabolic syndrome. Young (9 weeks old) ZFR males with matching control (Zucker lean littermates) were used in the experiment summarized in Table 3.

Table 3 shows that even at age 9 weeks, ZFR have significantly elevated body and liver weights, increased plasma triglycerides and cholesterol. However plasma glucose concentration remained at the control level despite substantially induced insulin levels. While lactolycopene (Inneov) did not reduce body weight in the lean rats, 8 day dietary treatment with lactolycopene lead to the noticeable decrease in body and liver weights of ZFR. Some reduction in liver weight has been also seen in the control animals (ZLR) treated with lactolycopene. The most drastic changes took place in the plasma triglyceride and cholesterol levels—both of them dropped respectively by 83.1% and 64.3% with lactolycopene treatment. There is also statistically significant decrease in the plasma insulin level of ZFR kept on lactolycopene diet.

As can be seen from Table 4, ZFR had an increased amount of SREBP-1c transcripts as well as elevated mRNAs for major lipogenic enzymes—FAS, SCD-1, ACL. Lactolycopene treatment diminished induction of lipogenic mRNAs, although that effect was partial, reflecting remaining hyperinsulinemia in ZFR on lactolycopene diet. Immunoblot results, presented in FIG. 2, showed that ZFR have significantly higher levels of SREBP-1 protein in liver nuclear extracts, and that amount was not changed with lactolycopene treatment.

The only mRNA that went down to the control value was fatty acid synthase. Hepatic ACL and SCD-1 mRNAs were reduced as much as two folds as compared to ZFR untreated group. These changes of lipogenic mRNAs explain reduction of plasma triglyceride level in ZFR treated with lactolycopene and confirm the data presented in the example 2.

Unchanged level of SREBP-2 is consistent with the normal values for cholesterologenic enzymes (HMG-CoA-R and HMG-CoA-S) in the livers of untreated ZFR and was confirmed by immunoblot data (see FIG. 2). Thus, hypercholesterolemia in young ZFR is rather attributable to diminished LDL reuptake in the liver, than activation of cholesterol biosynthesis in hepatocytes. Regardless phenotype, lactolycopene treatment reduced mRNA level for mayor transcriptional activator of cholesterol biosynthesis—SREBP-2 (˜2 fold). This finding supports the results from HepG2 and primary hepatocyte studies, presented as examples 1 and 2. However, SREBP-2 mRNA reduction had different effect on HMG-CoA-R and HMG-CoA-S transcripts in livers of diabetic and non-diabetic animals. Fatty rats showed significant decrease in both HMG-CoA-R and HMG-CoA-S mRNA levels, whereas lean littermates had those near control level.

There is also a noticeable induction of LDR-R protein, confirmed by immunoblot analysis in the livers of ZFR treated with lactolycopene (FIG. 1). This observation explains remarkable normalizing effect of lactolycopene diet on plasma lipid levels in ZFR. LDL-receptor turnover is regulated by SREBP-2 in animal liver. There is substantial upregulation of SREBP-2 nuclear form in the livers of ZFR treated with lactolycopene. Such an increase may explain the mechanism of hepatic LDL-receptor activation with lactolycopene.

Lactolycopene diet had no effect Cyp7-alpha mRNA, the enzyme responsible for cholesterol secretion into the bile. Zucker fatty rats are known to be a unique animal model displaying abnormally low bile cholesterol secretion rate. That problem develops as consequence of reduced rate of CYP-7 alpha transcription in their livers. Indeed, as can be seen from Table 4, CYP7a mRNA level is reduced in ZFR and remains at near the same level regardless of treatment performed.

Zucker Fatty Rats were chosen because of their extreme and rapidly forming phenotype, which resembles metabolic syndrome in humans and results in hyperinsulinaemia, weight gain, hyperlipidaemia and steatosis. All these characteristics were corrected at some extent by lactolycopene treatment.

However as can be seen from Table 3, 9 weeks old ZFR remain normoglycemic, despite of hyperinsulinaemia. This provides indication that animals used in the study have not developed yet activated gluconeogenesis, which is a key characteristic of type II diabetes, and an apparent feature of another closely related strain of Zucker rats—Zucker Diabetic Rats (ZDR). As a result of impaired (but still functional) insulin sensitivity, there is a significant reduction of insulin-sensitive mRNAs (PEPCK, G-6-Pase, IGFBP-1, IRS-2) in the livers of untreated ZFR. Therefore, such a preserved level of the insulin response in ZFR livers did not allow us to evaluate possible in vivo effect of lactolycopene on mechanisms gluconeogenesis and hepatic insulin sensitivity seen in rat primary hepatocyte study (example 2).

In the human hepatoma cell line HepG2 (see example 1), lactolycopene suppresses transcription of major gene, regulating cholesterol homeostasis—SREBP-2. This observation was confirmed using another type of cells—primary rat hepatocytes (see example 2). The effect of lactolycopene on SREBP-2 was also reproduced in Zucker Fatty Rats, whose plasma cholesterol and triglyceride levels dropped to the near control values after 8 days of treatment with 0.2% lactolycopene diet. Normalizing effect of lactolycopene on plasma lipid level is related, in our view, not only to the reduced levels of SREBP-2 in liver, but also to the enhanced levels of hepatic LDL-R (mRNA and protein) observed in the animals on lactolycopene diet. Therefore examples 1, 2 and 3 reveal, that lactolycopene affects cholesterol homeostasis at different levels—transcription of SREBP-2, regulation of LDL-receptor expression, influence on SCAP mRNA level and overall cholesterol synthesis rate.

It was unexpectedly found and reproduced in the independent study, that incubation HepG2 cells with lactolycopene leads to the repression of insulin-inducible gene-1 (INSIG-1). We have assumed that lactolycopene may regulate liver-specific insulin response in the cultured cells and in the liver. Negative effect of lactolycopene on INSIG-1 mRNA was also observed in rat primary hepatocytes, especially in presence of insulin. To our surprise, we also have observed that short-term dietary treatment with lactolycopene lowers body and liver weights, plasma insulin level in Zucker Fatty Rats and leads to normalization of plasma lipid profile.

The INNEOV lactolycopene formulation therefore has profound effect on multiple features of metabolic syndrome—reduction of plasma insulin level, plasma lipids, liver/body weight ratio, normalized transcription of lipogenic genes and increased LDL-receptor protein in livers of Zucker Fatty Rats treated with lactolycopene.

### EXAMPLE 4

### A. Effect of Lactolycopene Products on Plasma Lipid, Glucose and Insulin Levels

The effect of lactolycopene products on plasma lipid, glucose and insulin levels was investigated in human volunteers and the results shown in tables 5-11.

16 volunteers (8 male and 8 female) age 45-62, were selected for this pilot clinical trial. To 12 of them (6 male and 6 female) lactolycopene (INNEOV, L'Oréal (UK) Ltd, London), LL, was given, and to 4 of them (2 male and 2 female) Lyc-O-Mato™, LM, (Vita Healthcare) was given. There were two patients with diabetes in the former group and two patients with diabetes in the latter one. For these diabetes patients, in addition to the lipid level, concentration of glucose was also monitored. The concentration of insulin was also monitored in all 16 patients. LL was given to 3 patients—1 dragee twice daily with food; to the other 9 patients—1 dragee three times daily with food. LM was given in 3 capsules daily with food.

Serum samples were collected from the blood of the patients before the treatment and every two weeks after it had started. The treatment with both lycopene products lasted for two months.

The results of the influence of LL and LM on serum lipid parameters of these volunteers are presented in Tables 5, 6, 7 and 8. These results show that administration of either 2 or 3 dragees of LL (tables 5 and 6) or 3 capsules of LM per day (tables 7 and 8) caused a reduction in concentration of total cholesterol and triglycerides. Administration of LL only also caused a significant reduction (p<0.05) in the concentration of CH-LDL. Other lipid parameters of the volunteers were not significantly affected.

It was interesting to note the higher level of these lipids before the treatment was, the more profound the reduction of their concentration in serum was observed. For example, for patient No 9, after only two weeks of treatment by LL the level of total cholesterol was reduced almost by 36% from 247 to 158 mg/dL (table 5).

Comparison of the effect of administration of different preparations of lycopene showed that LL had a more profound effect than LM on the level of serum total cholesterol, LDL cholesterol and triglycerides.

In addition it was also important that the use of absorbable, i.e. bio-available, lycopene preparation either LL or LM formulation resulted in the reduction of serum glucose concentration in patients with an elevated level of it (tables 9 and 10).

It was interesting to observe that administration of LL or LM did not affect the physiological level of glucose in the non-hyperglycaemic patient serum but was able to reduce or normalise it in the patients with hyperglycaemia (numbers in bold).

Hyperglycaemic patients were those with a serum glucose concentration of greater than 6 mmol/l.

Comparison of the effect of administration of different preparations of lycopene showed that LL was able to reduce plasma glucose levels in patients with hyperglycaemia by an average of 28%, whereas LM was able to reduce this parameter by only 16%.

A similar effect of LL was observed with regard to insulin level. In patients with an elevated insulin level, two weeks of administration of the LL resulted in its normalisation (numbers in bold). However, this preparation did not affect the concentration of insulin in patients with normal physiological level of insulin (table 11).

### B. Effect of Lycopene Products on Body Mass in Humans

The effect of lycopene products on body mass was studied in a second pilot clinical trial involving 18 volunteers, 10 male and 8 female, aged 45-62. To 9 volunteers, 5 male and 4 female, 1 dragee of Lacto-Lycopene™ (INNEOV, L'Oreal (UK) Ltd, London), LL, was given three times daily with food. To the other 9, 5 male and 4 female, 1 capsule of Lyc-O-Mato™, LM, (Vita Healthcare) was given three times daily with food.

The trial lasted for two months and the volunteers' weight was measured before and after the trial. The results of the trial are presented in Table 12.

Administration of both LL and LM for two months resulted in body mass loss in 5 and 3 volunteers in each respective treatment group. However, the total mass loss in the whole LL group was 3 times more than in the LM group, i.e. 15 kg for the former and 5 kg for the latter. On average, each person in the LL group lost 1.7 kg and each person in the LM group lost 0.6 kg. It was interesting to note that the overweight volunteers benefited significantly more from this effect of LL. 4 out of 5 of them lost body mass between 2 and 5 kg. In the LM group, only 1 out of 5 overweight people lost body mass.

Lycopene treatment therefore reduces basic clinical features of metabolic syndrome (hypercholesterolemia, hypertriglyceridemia and hyperglycemia) in patients.

These clinical data support the experimental results which were observed in experiments in vitro and on animals, and indicate that absorbable and bio-available forms of lactolycopene preparations could be effective therapeutic products, which could be used in the treatment of metabolic syndrome, insulin resistance (including syndromes of severe insulin resistance), glucose tolerance, polycystic ovary syndrome (PCOS), hypertension, steatosis, chronic hepatitis, liver fibrosis, cirrhosis, and also for weight control and in the treatment of obesity.

### C. Effect of Statins on Serum Lipids

The effect of Lipitor-Liprimar (Atorvastatin) on serum lipid levels was investigated in human volunteers and the results shown in table 13.

Ten volunteers, 5 males and 5 females, aged 45-55 years old, were selected for this pilot clinical trial. 40 mg of Lipitor-Liprimar was given orally, with food, to each patient daily for two months.

Serum samples were collected from the blood of the patients before the treatment and after two months of treatment.

The results of the influence of Lipitor-Liprimar on serum lipid levels in these volunteers are presented in Table 13. The data presented in the table demonstrate that Lipitor-Liprimar, a HMG CoA reductase inhibitor, reduced CH-LDL better than LL (35% for former and 13.3% for the latter). However, lactolycopene reduces both total cholesterol and triglycerides to a lower level than the statin: total cholesterol (CH) reduction for Lipitor was 17% and for LL, it was 27%; triglyceride (TG) reduction for Lipitor was 22% and for LL, it was 34% (tables 6 and 13).

As well as being less effective at reducing serum lipid levels, statins are known to have some adverse effects in 0.5% to 2.0% of patients. For example, statins may cause elevated levels of transaminases or muscle pain, tenderness or weakness. These symptoms may be accompanied in some patients by fever or flu-like symptoms, abdominal pain or unexplained fatigue. Furthermore, liver disease and myositis are among contraindications for taking statins and they should not be prescribed during pregnancy. On the other hand, long-term administration of lactolycopene preparations has no known contraindications or side effects.

This, together with data presented above, provides indication that a lactolycopene formulation such as LL can be used as an alternative to statins in the treatment of various disorders including metabolic syndrome, insulin resistance, impaired glucose tolerance, hypertension, polycystic ovary syndrome, obesity, steatosis, chronic hepatitis and liver cirrhosis.

In addition, a lactolycopene formulation, such as LL, could be useful in combination with statins, allowing therapeutic doses of the latter to be reduced. This would result in fewer contraindications for statins and minimise their possible side-effects.

### D Additional Formulations

The effect of an isolated lactolycopene formulation (INDENA) on serum lipid levels was investigated in human volunteers and the results shown in table 15 and 16.

These results show that the lactolycopene formulation (INDENA) reduces serum lipid levels but has no significant effect on glucose levels in human plasma. However, it normalised concentrations of total cholesterol and triglycerides in serum of all subjects with their elevated levels. The higher level of these parameters was before the treatment the more significant reduction was observed. For example for subjects with total cholesterol above 250 mg/dL the reduction was in average 94 mg/dL, or more than 35%.

In subjects with initial concentration below 250 mg/dL but still above the normal level of 200 mg/dL, the reduction was in average 54 mg/dL, or 25%. A similar pattern was observed for triglycerides. A reduction of both parameters was observed even in subjects with physiologically normal level of these lipids—15% for the total cholesterol and 20% for triglycerides.

Less profound effects were observed for CH-HDL and CH-LDL concentrations. In 5 subjects, with reduced level of the former, 4 after treatment had normalised level of this lipid. Only in 2 out of 4 subjects, with initially elevated level of CH-LDL, had this parameter normalised.

### EXAMPLE 5

Incorporation of Lactolycopene into a Food Matrix

The recipe for one loaf of bread was:

yeast—¾ tsp, strong white flour—400 g, sugar—1 tsp, butter—15 g, milk powder—1 tsp, salt—1 tsp, water 280 ml, in which Lactolycopene (LL)(INNEOV) dragees were dissolved.

Four loaves were baked: one (the control) did not contain LL, the second contained 5 dragees of LL, the third—10 dragees of LL and the fourth—20 dragees of LL.

### Testing

Five volunteers who tested the bread blind-folded found no difference in texture or flavour between all four of these loaves. Furthermore, the antioxidant activity of the bread was tested.

This was done using an AtheroAbzyme™ ELISA kit (CTL, UK), following the protocol set out in the manufacturers' instructions. Bread comprising Lactolycopene had antioxidant activity, whereas the control bread did not (table 14).

### EXAMPLE 6

Incorporation of Lycopene into a Food Matrix

The recipe for a batch of four scones was:

self-raising flour—175 g, baking powder—1 tsp, pinch of salt, caster sugar—20 g, unsalted butter—37 g, milk—90 ml in which LL in the form of INNEOV dragees was dissolved.

Four batches of scones were baked: one (the control) did not contain LL, the second contained 0.5 dragee of LL per scone, the third—1 dragee of LL per scone and the fourth—2 dragees of LL per scone.

### Testing

Five volunteers who tested these scones blind-folded found no difference in texture or flavour between scones from all these four batches. Furthermore, the antioxidant activity of the scones was tested. This was done using an AtheroAbzyme™ ELISA kit (CTL, UK), following the protocol set out in the manufacturers instructions. Scones comprising Lactolycopene had antioxidant activity, whereas the control scones did not (table 14).

TABLE 1

| | −lactolycopene | | +lactolycopene | |
|---|---|---|---|---|
| mRNA | −Sterols | +Sterols | −Sterols | +Sterols |
| GAPDH | 17.73 | 17.69 | 17.60 | 17.54 |
| SREBP-1 | 1 | 1.03 | 0.89 | 1.37 |
| SREBP-2 | 1 | 0.47 | 0.58 | 0.38 |
| FPPS | 1 | 0.25 | 1.12 | 0.27 |
| HMG-CoA Syn | 1 | 0.10 | 1.02 | 0.07 |
| HMG-CoA Red | 1 | 0.18 | 1.06 | 0.14 |
| SCD-1 | 1 | 0.35 | 0.91 | 0.30 |
| FAS | 1 | 0.43 | 1.05 | 0.34 |
| ACC | 1 | 0.28 | 0.99 | 0.22 |
| SCAP | 1 | 0.66 | 0.66 | 0.90 |
| INSIG-1 | 1 | 0.61 | 0.34 | 0.56 |
| INSIG-2 | 1 | 1.68 | 1.16 | 1.19 |

Abbreviations:
GAPDH—glyceraldehhyde-3-phosphatase dehydrogenase;
SREBP-1—sterol regulatory element binding protein-1;
SREBP-2—sterol regulatory element binding protein-2;
FPPS—farnesyl diphosphate synthase;
HMG-CoA Syn—HMG-CoA synthase;
HMG-CoA Red—HMG-CoA Reductase;
SCD-1—stearoyl-CoA-desaturase-1;
FAS—fatty acid synthase;
ACC—acetyl CoA carboxylase;
SCAP—sterol cleavage activating protein;
INSIG-1—insulin-inducible gene-1;
INSIG-2—insulin-inducible gene-2.

TABLE 2

| | |
|---|---|
| −lactolycopene | +lactolycopene |

| mRNA | −Insulin | +Insulin | −Insulin | +Insulin |
|---|---|---|---|---|
| 36B4 | 24.90 | 24.29 | 24.03 | 24.01 |
| SREBP-1c | 1 | 56.50 | 3.80 | 27.50 |
| SREBP-2 | 1 | 0.92 | 0.75 | 0.46 |
| FAS | 1 | 4.04 | 1.33 | 1.40 |
| SCD-1 | 1 | 2.49 | 0.65 | 0.92 |
| ACL | 1 | 1.66 | 0.94 | 0.83 |
| PEPCK | 1 | 0.12 | 0.66 | 0.05 |
| IGFBP-1 | 1 | 0.03 | 0.73 | 0.02 |
| G-6-Pase | 1 | 0.90 | 0.70 | 0.62 |
| IRS-2 | 1 | 0.16 | 0.68 | 0.09 |
| INSIG-1 | 1 | 1.45 | 0.90 | 0.62 |
| INSIG-2 | 1 | 0.72 | 1.59 | 0.31 |
| Insulin-R | 1 | 0.70 | 1.22 | 0.21 |

Abbreviations:
36B4—acidic ribosomal phosphoprotein;
SREBP-1—sterol regulatory element binding protein-1;
SREBP-2—sterol regulatory element binding protein-2;
FAS—fatty acid synthase; SCD-1—stearoyl-CoA-desaturase-1;
ACL—ATP-citrate lyase,
PEPCK—Phosphoenolpyruvate carboxykinase,
IGFBP-1—insulin-like growth factor binding protein,
G-6-Pase—glucose-6-phosphatase;
IRS-2—insulin receptor substrate 2;
INSIG-1—insulin-inducible gene-1;
INSIG-2—insulin-inducible gene-2;
Insulin-R—insulin receptor.

TABLE 3

| | | | Chow + 0.2% | |
|---|---|---|---|---|
| | Chow | | lactolycopene | |
| Parameters | ZLR | ZFR | ZLR | ZFR |
| Body weight, g | 317.8 ± 25.18 | 419.9 ± 13.51 | 301.20 ± 9.57 | 368.37 ± 32.30 |
| Liver weight, g | 12.03 ± 1.93 | 19.05 ± 2.76 | 9.74 ± 0.91 | 14.16 ± 1.35 |
| LW/BW ratio | 0.037 ± 0.005 | 0.045 ± 0.005 | 0.032 ± 0.002 | 0.038 ± 0.002 |
| Plasma TG | 106.22 ± 68.96 | 1973.8 ± 328.3 | 50.87 ± 7.22 | 332.8 ± 107.26 |
| Plasma CH | 75.77 ± 18.86 | 235.70 ± 122.69 | 58.77 ± 3.95 | 83.75 ± 16.18 |
| Glucose | 127.00 ± 14.67 | 116.00 ± 13.92 | 104.00 ± 13.73 | 127.75 ± 14.93 |
| Insulin | 3.85 ± 1.20 | 16.30 ± 2.37 | 2.01 ± 0.70 | 11.19 ± 0.78 |

TABLE 4

| | |
|---|---|
| | 0.2% |
| Chow | lactolycopene Diet |

| mRNA | ZLR | ZFR | ZLR | ZFR |
|---|---|---|---|---|
| 36B4 | 22.49 | 22.63 | 22.45 | 22.26 |
| SREBP-1c | 1 | 2.78 | 1.35 | 2.24 |
| SREBP-2 | 1 | 0.92 | 0.52 | 0.48 |
| FAS | 1 | 3.52 | 1.32 | 1.15 |
| SCD-1 | 1 | 4.92 | 2.37 | 2.72 |
| ACL | 1 | 1.51 | 0.65 | 0.71 |
| PEPCK | 1 | 0.46 | 0.91 | 0.47 |
| IGFBP-1 | 1 | 0.25 | 0.61 | 0.59 |
| G-6-Pase | 1 | 0.40 | 0.46 | 0.46 |
| IRS-1 | 1 | 1.23 | 0.86 | 0.88 |
| IRS-2 | 1 | 0.46 | 0.84 | 0.33 |
| INSIG-2 | 1 | 1.22 | 0.89 | 0.66 |
| LDL-R | 1 | 1.09 | 0.76 | 1.69 |
| CYP-7α | 1 | 0.58 | 0.43 | 0.47 |
| INSIG-1 | 1 | 1.14 | 0.87 | 1.17 |
| BMG-CoA-S | 1 | 1.13 | 0.94 | 0.44 |
| HMG-CoA-R | 1 | 0.70 | 1.22 | 0.21 |

Abbreviations:
36B4—acidic ribosomal phosphoprotein;
SREBP-1—sterol regulatory element binding protein-1;
SREBP-2—sterol regulatory element binding protein-2;
FAS—fatty acid synthase;
SCD-1—stearoyl-CoA-desaturase-1;
ACL—ATP-citrate lyase;
PEPCK—Phosphoenolpyruvate carboxykinase;
IGFBP-1—insulin-like growth factor binding protein;
G-6-Pase—glucose-6-phosphatase;
IRS-1—insulin receptor substrate 1;
IRS-2—insulin receptor substrate 2;
INSIG 2—insulin-inducible gene-2;
LDL-R—low density lipoprotein receptor;
CYP-7α—7α-hydroxylase;
INSIG-1—insulin inducible gene 1;
HMG-CoA-S—HMG-CoA Synthase;
HMG-CoA-R—HMG-CoA Reductase;
INSIG-1—insulin-inducible gene;
INSIG-1—insulin-inducible gene-1.

TABLE 5

| | Daily | | Plasma lipids, in mg/dL** | | | | | |
|---|---|---|---|---|---|---|---|---|
| Patients | intake* | Gender | CH | TG | HDL-CH | LDL-CH | ApoA | ApoB |

before treatment

| | | | | | | | | |
|---|---|---|---|---|---|---|---|---|
| N°1 | 2 | f | 160 | 83 | 40 | 139 | 155 | 125 |
| N°2 | 3 | f | 259 | 141 | 45 | 128 | 320 | 128 |
| N°3 | 2 | m | 188 | 91 | 27 | 104 | 113 | 134 |
| N°4 | 3 | f | 192 | 115 | 37 | 112 | 201 | 101 |
| N°5 | 2 | m | 190 | 87 | 30 | 136 | 190 | 132 |
| N°6 | 3 | m | 182 | 127 | 40 | 198 | 184 | 101 |
| N°7 | 3 | f | 221 | 94 | 43 | 134 | 149 | 139 |
| N°8 | 3 | m | 208 | 83 | 30 | 146 | 127 | 127 |
| N°9 | 3 | m | 247 | 124 | 31 | 144 | 180 | 144 |
| N°10 | 3 | f | 234 | 144 | 37 | 140 | 200 | 130 |
| N°11 | 3 | f | 193 | 159 | 49 | 107 | 177 | 119 |
| N°12 | 3 | m | 201 | 90 | 53 | 142 | 117 | 92 |

| | | | | | | |
|---|---|---|---|---|---|---|
| mean | 206 | 112 | 38.5 | 135 | 176 | 123 |

after 2 weeks of treatment

| | | | | | | | | |
|---|---|---|---|---|---|---|---|---|
| N°1 | 2 | f | 141 | 72 | 43 | 122 | 153 | 119 |
| N°2 | 3 | f | 209 | 127 | 45 | 125 | 287 | 129 |
| N°3 | 2 | m | 175 | 87 | 34 | 108 | 110 | 132 |
| N°4 | 3 | f | 186 | 77 | 40 | 130 | 200 | 100 |
| N°5 | 2 | m | 189 | 82 | 41 | 145 | 188 | 130 |
| N°6 | 3 | m | 151 | 111 | 40 | 146 | 180 | 106 |
| N°7 | 3 | f | 181 | 69 | 45 | 132 | 140 | 130 |
| N°8 | 3 | m | 201 | 77 | 37 | 136 | 122 | 125 |
| N°9 | 3 | m | 158 | 113 | 40 | 107 | 150 | 140 |
| N°10 | 3 | f | 220 | 109 | 42 | 137 | 181 | 125 |
| N°11 | 3 | f | 152 | 130 | 49 | 107 | 165 | 117 |
| N°12 | 3 | m | 149 | 80 | 57 | 130 | 115 | 90 |

| | | | | | | |
|---|---|---|---|---|---|---|
| mean | 176 | 94.5 | 42.7 | 127 | 166 | 120 |

after 1 month of treatment

| | | | | | | | | |
|---|---|---|---|---|---|---|---|---|
| N°1 | 2 | f | 125 | 70 | 50 | 120 | 150 | 110 |
| N°2 | 3 | f | 189 | 120 | 49 | 126 | 269 | 127 |
| N°3 | 2 | m | 170 | 69 | 33 | 106 | 107 | 129 |
| N°4 | 3 | f | 161 | 74 | 49 | 125 | 197 | 97 |
| N°5 | 2 | m | 150 | 61 | 45 | 126 | 168 | 121 |
| N°6 | 3 | m | 134 | 100 | 50 | 136 | 170 | 100 |
| N°7 | 3 | f | 179 | 67 | 44 | 130 | 137 | 131 |
| N°8 | 3 | m | 178 | 78 | 40 | 133 | 123 | 120 |
| N°9 | 3 | m | 154 | 96 | 40 | 107 | 147 | 137 |
| N°10 | 3 | f | | | | | | |
| N°11 | 3 | f | 151 | 99 | 47 | 105 | 160 | 117 |
| N°12 | 3 | m | 147 | 80 | 55 | 129 | 117 | 88 |

| | | | | | | |
|---|---|---|---|---|---|---|
| mean | 158 | 83.1 | 49.4 | 121 | 175 | 116 |

after 6 weeks of treatment

| | | | | | | | | |
|---|---|---|---|---|---|---|---|---|
| N°1 | 2 | f | 125 | 72 | 50 | 119 | 157 | 111 |
| N°2 | 3 | f | 176 | 119 | 52 | 120 | 188 | 128 |
| N°3 | 2 | m | 167 | 65 | 37 | 105 | 106 | 127 |
| N°4 | 3 | f | 160 | 67 | 48 | 120 | 192 | 95 |
| N°5 | 2 | m | 149 | 60 | 45 | 123 | 167 | 120 |
| N°6 | 3 | m | 136 | 84 | 49 | 130 | 165 | 100 |
| N°7 | 3 | f | 177 | 66 | 45 | 130 | 134 | 130 |
| N°8 | 3 | m | 144 | 70 | 42 | 132 | 120 | 120 |
| N°9 | 3 | m | 150 | 101 | 41 | 108 | 141 | 132 |
| N°10 | 3 | f | 180 | 140 | 40 | 137 | 181 | 127 |
| N°11 | 3 | f | 132 | 89 | 45 | 100 | 151 | 114 |
| N°12 | 3 | m | 145 | 71 | 54 | 120 | 117 | 87 |

| | | | | | | |
|---|---|---|---|---|---|---|
| mean | 153 | 84.5 | 45.7 | 120 | 151 | 116 |

after 8 weeks of treatment

| | | | | | | | | |
|---|---|---|---|---|---|---|---|---|
| N°1 | 2 | f | 129 | 75 | 50 | 118 | 144 | 110 |
| N°2 | 3 | f | 170 | 93 | 50 | 115 | 176 | 118 |
| N°3 | 2 | m | 147 | 65 | 40 | 103 | 100 | 127 |
| N°4 | 3 | f | 161 | 69 | 47 | 121 | 180 | 96 |
| N°5 | 2 | m | 150 | 60 | 45 | 120 | 164 | 119 |
| N°6 | 3 | m | 143 | 84 | 47 | 127 | 166 | 105 |
| N°7 | 3 | f | 179 | 67 | | 130 | | |
| N°8 | 3 | m | 178 | 78 | | 132 | | |
| N°9 | 3 | m | 154 | 96 | | 108 | | |
| N°10 | 3 | f | 152 | 74 | | 137 | | |
| N°11 | 3 | f | 151 | 99 | | 100 | | |
| N°12 | 3 | m | 147 | 80 | | 120 | | |

| | | | | | | |
|---|---|---|---|---|---|---|
| mean | 150 | 74.3 | 46.5 | 119 | 155 | 112 |

*number of “INNEOV” dragees (20 mg of Lacto-Lycopene per one dragee);
**CH—total cholesterol, TG—triglycerides;

TABLE 6
Mean serum lipid concentration, in mg/dL

| before treatment | | | after 2 months of treatment | | |
|---|---|---|---|---|---|
| CH | TG | CH-LDL | CH | TG | CH-LDL |
| 206 ± | 112 ± 8.9 | 135 ± 6.0 | 150 ± 4.1* | 74.3 ± 3.8* | 119 ± 3.3* |
| 8.6 | | | p < 0.001 | p < 0.002 | p < 0.05 |
| 100% | 100% | 100% | 73% | 66% | 88% |

*statistically significant

TABLE 7

| | Daily | | Plasma lipids, in mg/dL** | | | | | |
|---|---|---|---|---|---|---|---|---|
| Patients | intake* | Gender | CH | TG | HDL-CH | LDL-CH | ApoA | ApoB |

before treatment

| | | | | | | | | |
|---|---|---|---|---|---|---|---|---|
| 13 | 3 | f | 157 | 117 | 30 | 122 | 100 | 77 |
| 14 | 3 | f | 161 | 119 | 31 | 140 | 120 | 80 |
| 15 | 3 | m | 160 | 108 | 47 | 105 | 117 | 74 |
| 16 | 3 | m | 191 | 128 | 48 | 114 | 175 | 97 |
| 17 | 3 | f | 184 | 106 | | 85 | | |
| 18 | 3 | f | 185 | 126 | | 120 | | |
| 19 | 3 | f | 168 | 190 | | 92 | | |
| 20 | 3 | m | 197 | 95 | | 72 | | |
| 21 | 3 | m | 204 | 104 | | 115 | | |
| 22 | 3 | m | 220 | 120 | | 89 | | |

after 2 weeks of treatment

| | | | | | | | | |
|---|---|---|---|---|---|---|---|---|
| 13 | 3 | f | 150 | 102 | 30 | 120 | 100 | 76 |
| 14 | 3 | f | 160 | 117 | 32 | 137 | 119 | 79 |
| 15 | 3 | m | 141 | 97 | 48 | 92 | 102 | 71 |
| 16 | 3 | m | 156 | 98 | 48 | 79 | 145 | 82 |

after 4 weeks of treatment

| | | | | | | | | |
|---|---|---|---|---|---|---|---|---|
| 13 | 3 | f | 140 | 95 | 33 | 118 | 90 | 75 |
| 14 | 3 | f | 145 | 85 | 37 | 125 | 110 | 73 |
| 15 | 3 | m | 140 | 85 | 47 | 90 | 100 | 70 |
| 16 | 3 | m | 157 | 97 | 46 | 79 | 143 | 80 |

after 8 weeks of treatment

| | | | | | |
|---|---|---|---|---|---|
| 13 | 3 | f | 141 | 80 | 117 |
| 14 | 3 | f | 143 | 72 | 120 |
| 15 | 3 | m | 140 | 92 | 90 |
| 16 | 3 | m | 152 | 96 | 78 |
| 17 | 3 | f | 160 | 98 | 78 |
| 18 | 3 | f | 160 | 92 | 111 |
| 19 | 3 | f | 179 | 170 | 89 |
| 20 | 3 | m | 160 | 71 | 73 |
| 21 | 3 | m | 170 | 95 | 90 |
| 22 | 3 | m | 180 | 94 | 91 |

| | | | |
|---|---|---|---|
| mean | 159 | 96 | 94 |

*number of capsules given daily (15 mg of lycopene per one capsule)
**CH—total cholesterol, TG—triglycerides;

TABLE 8
Mean serum lipid concentration, in mg/dL

| before treatment | | | after 2 months of treatment | | |
|---|---|---|---|---|---|
| CH | TG | CH-LDL | CH | TG | CH-LDL |
| 183 ± | 121 ± 6.8 | 105 ± 7.2 | 159 ± 4.6* | 96.0 ± 6.2* | 94 ± 5.8 |
| 6.9 | | | p < 0.05 | p < 0.05 | p > 0.05 |
| 100% | 100% | 100% | 87% | 79% | 90% |

*statistically significant

TABLE 9

Concentration of glucose in patient
serum, in mmol/l

| | | after 2 months of |
|---|---|---|
| Patients | before treatment | treatment |
| 7 | 5.9 | 5.9 |
| 8 | 8.4 (100%) | 5.9 (70%) |
| 9 | 8.7 (100%) | 7.9 (91%) |
| 10 | 9.5 (100%) | 6.5 (68%) |
| 11 | 10.2 (100%) | 7.7 (75%) |
| 12 | 12.3 (100%) | 8.1 (66%) |

In patients with hyperglycaemia, mean reduction of glucose by 28%

TABLE 10

Concentration of glucose in patient
serum, in mmol/l

| | | after 2 months of |
|---|---|---|
| Patient ID | before treatment | treatment |
| 13 | 3.9 | 5.4 |
| 14 | 6.4 (100%) | 5.2 (81%) |
| 15 | 7.0 (100%) | 6.3 (90%) |
| 16 | 8.4 (100%) | 6.4 (76%) |
| 17 | 10 (100%) | 8.4 (84%) |
| 18 | 14 (100%) | 12.2 (87%) |

In patients with hyperglycaemia, mean reduction of glucose by 16%

TABLE 11

Concentration of insulin
in patient serum, in μg/ml

| Patient | Before treatment | 2 weeks of the treatment |
|---|---|---|
| 1 | 13 | 11 |
| 12 | 31 | 7.4 |

TABLE 12

| LL | | | LM | | |
|---|---|---|---|---|---|
| weight | weight | weight | weight | weight | weight |
| before | after | loss | before | after | loss |
| (kg) | (kg) | (kg) | (kg) | (kg) | (kg) |
| 130 | 125 | −5 | 134 | 132 | −2 |
| 100 | 97 | −3 | 105 | 106 | +1 |
| 100 | 100 | 0 | 103 | 103 | 0 |
| 94 | 91 | −3 | 96 | 96 | 0 |
| 94 | 92 | −2 | 92 | 92 | 0 |
| 90 | 90 | 0 | 85 | 83 | −2 |
| 89 | 89 | 0 | 82 | 82 | 0 |
| 82 | 82 | 0 | 80 | 78 | −2 |
| 68 | 66 | −2 | 72 | 72 | 0 |

| | | | |
|---|---|---|---|
| overweight | effect in | overweight | effect in |
| >92 kg | 4 out of 5 | >92 kg | 1 out of 5 |

| | |
|---|---|
| weight loss 15 kg per 9 | weight loss 5 kg per 9 |
| persons | persons |
| or 1.7 kg per person | or 0.6 kg per person |

TABLE 13

| | | | | |
|---|---|---|---|---|
| Patients | CH | TG | CH-LDL | CH-HDL |

Before treatment

| | | | | |
|---|---|---|---|---|
| 19 | 245 | 179 | 149 | 32 |
| 20 | 146 | 172 | 155 | 40 |
| 21 | 214 | 147 | 140 | 39 |
| 22 | 206 | 140 | 156 | 48 |
| 23 | 208 | 106 | 175 | 37 |
| 24 | 219 | 90 | 100 | 44 |
| 25 | 224 | 175 | 160 | 40 |
| 26 | 284 | 169 | 170 | 43 |
| 27 | 277 | 207 | 180 | 45 |
| 28 | 171 | 169 | 147 | 42 |
| mean | 219 | 145 | 153 | 41 |

After 2 months of treatment*

| | | | | |
|---|---|---|---|---|
| 19 | 200 | 120 | 120 | 38 |
| 20 | 140 | 136 | 145 | 42 |
| 21 | 176 | 99 | 110 | 42 |
| 22 | 188 | 98 | 146 | 47 |
| 23 | 197 | 87 | 140 | 39 |
| 24 | 200 | 90 | 100 | 43 |
| 25 | 170 | 133 | 130 | 42 |
| 26 | 184 | 105 | 110 | 49 |
| 27 | 209 | 172 | 130 | 47 |
| 28 | 154 | 96 | 120 | 43 |
| mean | 182 | 113 | 100 | 43 |

*Lipitor was taken 40 mg daily, orally.

TABLE 14

| | Anti-oxidant activity of 1 g of the food matrix | | | | |
|---|---|---|---|---|---|
| | containing: | | | | |
| Food | | 10⁻⁸M of | 10⁻⁷M of | 10⁻⁶M of | 10⁻⁵M of |
| Matrix | control | LL | LL | LL | LL |
| Loaf | 0 | 0 | 14% | 15% | 22% |
| Scone | 0 | 0 | 9% | 29% | 100% |

TABLE 15

| | Total cholesterol | | | LDL-cholesterol | | | HDL-cholesterol | | | Triglycerides | |
|---|---|---|---|---|---|---|---|---|---|---|---|
| N°* | before | after | N°* | before | after | N°* | before | after | N°* | before | after |
| 172 | 248 | 183 | 168 | 183 | 170 | 171 | 39 | 43 | 168 | 209 | 127 |
| 179 | 240 | 151 | 169 | 169 | 167 | 168 | 33 | 39 | 169 | 180 | 146 |
| 168 | 254 | 180 | 162 | 180 | 132 | 162 | 34 | 42 | 162 | 205 | 107 |
| 169 | 240 | 200 | 167 | 167 | 110 | 167 | 37 | 45 | | | |
| 162 | 290 | 160 | | | | 174 | 37 | 43 | | 198 ± 10.4 | 126 ± 11.6 |
| 167 | 298 | 140 | | 175 ± 5 | 145 ± 17 | | | | | (n = 3) | (n = 3) |
| 192 | 284 | 184 | | (n = 4) | (n = 4) | | 36 ± 1.3 | 42 ± 1.0 | | | |
| | | | | | | | (n = 5) | (n = 5) | | p < 0.001 | |
| | 265 ± 11.4 | 171 ± 9.1 | | | p > 0.05 | | | | | Δ = 72 mg/dL | |
| | (n = 7) | (n = 7) | | | | | | | | | |
| | | | | | | | | | 18 | 145 | 77 |
| | | | | | | | | | 167 | 165 | 102 |
| | | | | | | | | | 192 | 163 | 128 |
| | | | | | | | | | 170 | 154 | 80 |
| | | | | | | | p < 0.01 | | | 157 ± 5.2 | 97 ± 13 |
| | p < 0.001 | | | | | | Δ = 6 mg/dL | | | (n = 4) | (n = 4) |
| | Δ = 94 mg/dL | | | | | | | | | p < 0.001 | |
| 171 | 207 | 149 | | | | | | | | Δ = 60 mg/dL | |
| 182 | 209 | 170 | | | | | | | | | |
| 18 | 226 | 160 | | | | | | | | | |
| | 214 ± 6.7 | 160 ± 6.1 | | | | | | | | | |
| | (n = 3) | (n = 3) | | | | | | | | | |
| | p < 0.001 | | | | | | | | | | |
| | Δ = 54 mg/dL | | | | | | | | | | |
| 170 | 192 | 180 | 171 | 110 | 111 | 172 | 44 | 44 | 174 | 121 | 88 |
| 174 | 189 | 138 | 182 | 120 | 115 | 179 | 45 | 49 | 181 | 105 | 90 |
| 181 | 190 | 180 | 18 | 101 | 100 | 182 | 44 | 46 | 185 | 108 | 74 |
| 185 | 198 | 159 | 192 | 150 | 144 | 169 | 40 | 41 | 171 | 94 | 80 |
| | | | 170 | 134 | 121 | 18 | 40 | 48 | 172 | 88 | 90 |
| | 192 ± 2.0 | 164 ± 11.4 | 174 | 110 | 100 | 192 | 40 | 43 | 179 | 103 | 90 |
| | (n = 4) | (n = 4) | | | | 170 | 40 | 45 | 182 | 135 | 102 |

| | | | 121 ± 8.0 | 115 ± 6.4 | 180 | 47 | 43 | | |
|---|---|---|---|---|---|---|---|---|---|
| Z | | | | | 181 | 43 | 44 | 108 ± 6.1 | 88 ± 3.2 |
| | p < 0.05 | | (n = 6) | (n = 6) | 185 | 40 | 44 | (n = 7) | (n = 7) |
| | Δ = 28 mg/dL | | | p > 0.05 | | | | | |
| | | | | | | 42.3 | 44.7 | p < 0.05 | |
| | | 172 | 97 | 85 | | (n = 10) | (n = 10) | Δ = 20 mg/dL | |
| | | 179 | 87 | 83 | | | p > 0.05 | | |
| | | 181 | 93 | 88 | | | | | |
| | | 185 | 79 | 70 | | | | | |
| | | | 89 ± 4.3 | 81.5 ± 4.2 | | | | | |
| | | | (n = 4) | (n = 4) | | | | | |
| | | | | p > 0.05 | | | | | |

In bold abnormal hyper- or hypo- levels of lipid parameters.
*Patient number.

TABLE 16

| | |
|---|---|
| before | after 2 months |

| | | 2 hours | | 2 hours |
|---|---|---|---|---|
| | fasting | after | fasting | after |
| Patient | level of | glucose | level of | glucose |
| ID | glucose | intake | glucose | intake |
| 168 | 9.3 | 12 | 7.8 | 12 |
| 169 | 4.5 | — | 5.5 | — |
| 170 | 5.1 | — | 5.4 | — |
| 18 | 5.4 | — | 5.6 | — |
| 179 | 5.4 | — | 5.3 | — |
| 182 | 5.6 | — | 6.6 | 8.1 |
| 162 | 6.9 | — | 6.4 | 7.9 |
| 167 | 5.3 | — | 5.5 | — |
| 171 | 6 | — | 6.1 | — |
| 172 | 5.7 | — | 5.6 | — |
| 174 | 4.7 | — | 5.3 | — |
| 181 | 5.4 | — | 5.4 | — |
| 185 | 6 | 8.1 | 6 | 8 |

1. A method of treating metabolic dysfunction comprising;
administering a lycopene compound in a therapeutically effective amount to an individual in need thereof.

2. A method according to claim 1 wherein the lycopene compound is lycopene.

3. A method according to claim 1 wherein the metabolic dysfunction is selected from the group consisting of
obesity, insulin resistance, reduced glucose tolerance, polycystic ovary syndrome, hypertension, steatosis, chronic hepatitis, liver fibrosis, cirrhosis, and metabolic syndrome.

4. A method according to claim 1 wherein the lycopene compound is formulated with whey protein.

5. A method according to claim 1 wherein the lycopene compound is formulated with oil.

6. A method according to claim 1 wherein the lycopene compound is formulated with one or more isoflavones.

7. A method according to claim 1 wherein the lycopene compound is comprised in a pharmaceutical composition with a pharmaceutically acceptable excipient.

8. A method according to claim 1 wherein the lycopene compound is comprised in a food product.

9. A method according to claim 8 wherein the food product is a bread, a cereal, a biscuit, butter, a spread, cheese, yogurt or a beverage.

10. A method according to claim 1 wherein the lycopene compound is for administration in combination with a statin.

11. A method according to claim 1 comprising administering the lycopene compound in combination with a statin.

12-22. (canceled)

23. A method of treating of a condition associated with metabolic dysfunction comprising:
administering a lycopene compound in a therapeutically effective amount to an individual in need thereof.

24. A method according to claim 23 wherein the lycopene compound is lycopene.

25. A method according to claim 23 wherein the a condition associated with metabolic dysfunction is selected from the group consisting of raised blood pressure, hypertriglyceridaemia, hypercholesterolaemia, high LDL cholesterol and/or low HDL cholesterol and microalbuminuria.

26. A method according to claim 23 wherein the lycopene compound is formulated with whey protein.

27. A method according to claim 23 wherein the lycopene compound is formulated with oil.

28. A method according to claim 23 wherein the lycopene compound is formulated with isoflavones.

29. A method according to claim 23 wherein the lycopene compound is comprised in a pharmaceutical composition with a pharmaceutically acceptable excipient.

30. A method according to claim 23 wherein the lycopene is comprised in a food product.

31. A method according to claim 30 wherein the food product is a bread, a cereal, a biscuit, butter, a spread, cheese, yogurt or a beverage.

32. A method according to claim 23 wherein the lycopene compound is for administration with a statin.

33. A method according to claim 23 comprising administering the lycopene compound in combination with a statin.

34-44. (canceled)

45. A kit containing a lycopene compound and a statin for use in combination to treat metabolic dysfunction.

46. A kit according to claim 45 wherein the lycopene compound is lycopene.

47. A kit according to claim 45 wherein the metabolic dysfunction is selected from the group consisting of
obesity, insulin resistance, reduced glucose tolerance, polycystic ovary syndrome, hypertension, steatosis, chronic hepatitis, liver fibrosis, cirrhosis, and metabolic syndrome.

48. A kit according to claim 45 wherein the lycopene compound is formulated with a whey protein.

49. A kit according to claim 45 wherein the lycopene compound is formulated with oil.

50. A kit according to claim 45 wherein the lycopene compound is formulated with isoflavones.

51. A kit according to claim 45 wherein the lycopene compound is comprised in a pharmaceutical composition with a pharmaceutically acceptable excipient.

52. A kit containing a lycopene compound and a statin for use in combination to treat a condition associated with metabolic dysfunction.

53. A kit according to claim 52 wherein the lycopene compound is lycopene.

54. A kit according to claim 52 wherein the condition is selected from the group consisting of
obesity, insulin resistance, reduced glucose tolerance, polycystic ovary syndrome, hypertension, steatosis, chronic hepatitis, liver fibrosis, cirrhosis, and metabolic syndrome.

55. A kit according to claim 52 wherein the lycopene compound is formulated with a whey protein.

56. A kit according to claim 52 wherein the lycopene compound is formulated with oil.

57. A kit according to claim 52 wherein the lycopene compound is formulated with isoflavones.

58. A kit according to claim 52 wherein the lycopene compound is comprised in a pharmaceutical composition with a pharmaceutically acceptable excipient.

59-76. (canceled)

77. A food product comprising a lycopene compound for treating metabolic dysfunction, wherein the lycopene compound is not naturally present in the food product.

78. A food product according to claim 77 wherein the food product is a bread, a cereal, a biscuit, butter, a spread, cheese, yogurt or a beverage.

79. A food product according to claim 77 wherein the lycopene compound is lycopene.

80. A food product according to claim 77 wherein the metabolic dysfunction is selected from the group consisting of: obesity, insulin resistance, reduced glucose tolerance, polycystic ovary syndrome, hypertension, steatosis, chronic hepatitis, liver fibrosis, cirrhosis, and metabolic syndrome.

81. The food product of claim 77 further comprising a statin.

82. A method of making a food product for treating metabolic dysfunction comprising
(i) providing a food product ingredient,
(ii) mixing a lycopene compound with said ingredient
(iii) formulating said ingredient and said lycopene compound into a food product.

83. A method according to claim 82 wherein the food product is a bread, a cereal, a biscuit, butter, a spread, cheese, yogurt or a beverage.

84. A method according to claim 82 wherein the lycopene compound is lycopene.

85. A method according to claim 82 wherein the metabolic dysfunction is selected from the group consisting of:
obesity, insulin resistance, reduced glucose tolerance, polycystic ovary syndrome, hypertension, steatosis, chronic hepatitis, liver fibrosis, cirrhosis, and metabolic syndrome.
